(19) United States
(12) Patent Application Publication
(10) Pub. No.: US 2012/0046573 A1
(43) Pub. Date: Feb. 23, 2012

(54) MID-FLOW APPLICATOR

(76) Inventors: Peter Bowman Magill, North Huntingdon, PA (US); Dolores Magill, North Huntingdon, PA (US)

(21) Appl. No.: 12/858,275

(22) Filed: Aug. 17, 2010

(51) Int. Cl. A61M 1/00

(52) U.S. Cl. 600/574

(57) ABSTRACT

A mid-flow applicator to assist in female urine collection comprising: a urine reservoir, where a first side funnel, a second side funnel, a back catch and a pelvic shield lip surround said reservoir; a back lip on each side of the back catch; a pelvic shield lip extending upwardly from the pelvic shield; a front lip on each side of the pelvic shield lip; and an opening at the bottom of the reservoir. The applicator further includes a mechanism to attach the opening to a urine sample cup. The mechanism to attach may include a screw, matching lip seal and a slip on attachment. The reservoir, funnels, back catch and pelvic shield are uniquely configured to encourage the flow of urine to the opening and into an attached urine sample cup.

### BACKGROUND OF THE INVENTION

1. Field of Invention

The present invention relates to a female urine collection device to assist in the collection of urine from a female.

2. Description of Related Art

A urine collection device allows for the collection of urine for analysis or a simple means for the collection of urine when a toilet is not available such as in an aircraft or a spacecraft. Various devices have been developed to assist in the collection of urine from a female due to the particularity of the female anatomy. When a female urinates the urine stream is not visible to the urinator therefore attempting to urinate in a small cup can cause several problems. A female's urine will normally splash, overflow or require maneuvering to ensure the urine collection is successful. The prior art includes female urine collection systems that implement various funnels that provide a means to relieve female urination.

One example of a female urine collector is disclosed in U.S. Pat. No. 6,537,262 that discloses a horizontally accordion-pleated, rectangular-shaped urine collection receptacle with a tube outlet, tube housing a manually operated tube valve disposed to direct urine flow either to a sample reservoir or a urine collector. It would be advantageous to have a urine collection device that may be attached to or screwed onto a urine sample cup therefore providing a collection device that is disposable and easily integrated with a urine sample collector. The collection device alleviates some of the problems and drawbacks with female urine collection. Further it would be advantageous to simply insert a collection device onto a standard urine sample cup for use with female urination.

### SUMMARY OF THE INVENTION

The present invention relates a mid-flow applicator to assist in female urine collection comprising: a urine reservoir, where said a first side funnel, a second side funnel, a back catch and a pelvic shield lip surround said reservoir; a back lip on each side of the back catch; a pelvic shield lip extending upwardly from the pelvic shield; a front lip on each side of the pelvic shield lip; and an opening at the bottom of the reservoir. The applicator further includes a means to attach the opening to a urine sample cup. The means to attach may include a screw on means, matching lip seal and a slip on attachment. The reservoir, funnels, back catch and pelvic shield are uniquely configured to encourage the flow of urine to the opening and into an attached urine sample cup.

### BRIEF DESCRIPTION OF DRAWINGS

FIG. 1 depicts a mid-flow applicator according to the present invention.

### DETAILED DESCRIPTION

The present invention relates to a mid-flow applicator according to the present invention. The mid-flow applicator provides a female urine collection device that is easily integrated with a conventional urine sample cup. The urine collection device enables a woman to provide a urine sample in a manner that avoids splashing, overflowing, contamination and other problems that may occur with the use of a conventional urine cup. The mid-flow applicator according to the present invention prevents the dripping of urine on to the floor or toilet seat and inserts onto a urine sample cup to collect the sample directly through the applicator. The applicator is compact and disposable and easy to use for urine collection.

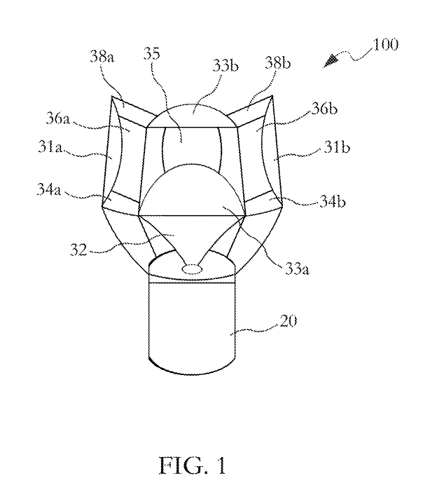

In reference to FIG. 1, a Mid-Flow Applicator 100 according to the present invention is depicted. The Mid-Flow Applicator 100 is attached to a urine sample Cup 20. The Flow Applicator 100 includes various components that assist in the collection of urine into the sample Cup 20. As depicted the mid-flow applicator 100 includes urine Reservoir 35 at the center of the device. The urine Reservoir 35 is surrounded by Side Funnels 36a, 36b, Back Lip 38a, 38b, Front Lips 34a, 34b. A back catch 33b is also provided to the rear of the urine Reservoir 35. A pelvic shield 32 with pelvic shield lip 33a is provided at the front portion of the urine reservoir.

The shape and configuration of the flow applicator allows for an effective and clean collection of female urine into the urine sample cup. The flow applicator includes a means of attaching the applicator to the sample cup. The means of securing the applicator to the sample cup 20 includes a screw on, matching lip seal, or slip on abutting surfaces connection. The mid-flow applicator 100 assists with the collection of urine within the Reservoir 35 and where the urine then flows through an opening provided at the connection point of the applicator to the sample cup 20. The mid-flow applicator is compact, easy to use and produced with disposable material. The wide breadth of the applicator with collapsible side shields ensures for the accurate control collection of the urine sample. The instant invention has been shown and described in what it considers to be the most practical and preferred embodiments. It is recognized, however, that departures may be made there from within the scope of the invention and that obvious modifications will occur to a person skilled in the art.

1. A mid-flow applicator to assist in female urine collection comprising:
a. a urine reservoir, where said a first side funnel, a second side funnel, a back catch and a pelvic shield lip surround said reservoir;
b. a back lip on each side of the back catch;
c. a pelvic shield lip extending upwardly from the pelvic shield;
d. a front lip on each side of the pelvic shield lip;
e. a first side lip at the top of the first side funnel and a second side lip at the top of the second side funnel; and
f. an opening at the bottom of the reservoir.

2. The applicator according to claim 1 where further including a means to attach the opening to a urine sample cup.

3. The applicator according to claim 2, where the means to attach includes at least one of a screw on means, matching lip seal and a slip on attachment.

4. The applicator according to claim 1 where the reservoir, funnels, back catch and pelvic shield are configured to encourage the flow of urine to the opening.
